(19) United States
(12) Patent Application Publication
(10) Pub. No.: US 2025/0114032 A1
(43) Pub. Date: Apr. 10, 2025

(54) DETECTION DEVICE

(71) Applicants: Japan Display Inc., Tokyo (JP); National University Corporation Tokyo Medical and Dental University, Tokyo (JP)

(72) Inventors: Yuji ONO, Tokyo (JP); Kei TAMURA, Tokyo (JP); Hiromi MITANI, Toyko (JP); Hiroko OHMORI, Tokyo (JP); Takashi ONO, Tokyo (JP); Hiroaki KIRIMOTO, Tokyo (JP)

(21) Appl. No.: 18/905,696

(22) Filed: Oct. 3, 2024

(30) Foreign Application Priority Data
Oct. 4, 2023 (JP) 2023-172697

(51) Int. Cl. A61B 5/00; A61B 5/389

(52) U.S. Cl. CPC A61B 5/4557; A61B 5/0002; A61B 5/389; A61B 5/682; A61B 5/743; A61B 2562/0247

(57) ABSTRACT

According to an aspect, a detection device includes: a force sensor disposed at a mouthpiece; a myoelectric sensor attachable to a human cheek; and a controller configured to perform output in which an output of the force sensor is synchronized with an output of the myoelectric sensor.

### CROSS-REFERENCE TO RELATED APPLICATION

This application claims the benefit of priority from Japanese Patent Application No. 2023-172697 filed on Oct. 4, 2023, the entire contents of which are incorporated herein by reference.

### BACKGROUND

### 1. Technical Field

What is disclosed herein relates to a detection device.

### 2. Description of the Related Art

Methods for detecting human bruxism are known, such as a method using a mouthpiece with a force sensor (refer to, for example, Japanese Patent No. 6634567) and a method using a myoelectric sensor (refer to, for example, Japanese Patent No. 6618482).

Both the method only using a force sensor and the method only using a myoelectric sensor may fail to determine whether data acquired from the sensor is data caused by bruxism or data not caused by bruxism, in some cases. For example, with the method using the force sensor, it is difficult to distinguish between force caused by bruxism and force caused by tongue or lips touching the force sensor. The myoelectric sensor sometimes produces an output similar to that caused by bruxism when the eyelids were tightly closed.

For the foregoing reasons, there is a need for a detection device that can output data allowing data caused by bruxism and data not caused by bruxism to be distinguished from each other more accurately.

### SUMMARY

According to an aspect, a detection device includes: a force sensor disposed at a mouthpiece; a myoelectric sensor attachable to a human cheek; and a controller configured to perform output in which an output of the force sensor is synchronized with an output of the myoelectric sensor.

### BRIEF DESCRIPTION OF THE DRAWINGS

FIG. 1 is a block diagram illustrating a main configuration of a detection system having a detection device;

FIG. 2 is a diagram illustrating an exemplary outer shape of the detection device;

FIG. 3 is a schematic exploded perspective view of a main part configuration example of a first section;

FIG. 4 is a schematic side view illustrating a mechanism of a first part and a second part bonded together;

FIG. 5 is a schematic plan view of a main part configuration example of the first section;

FIG. 6 is a schematic diagram illustrating an example of attachment of the detection device to a human;

FIG. 7 is a schematic diagram illustrating the arrangement of the first section in the mouth illustrated in FIG. 6;

FIG. 8 is a schematic diagram illustrating an attachment position of first, second, and third electrodes;

FIG. 9 is a graph illustrating an example of the relation in time sequence between human bite force indicated by output of a force sensor and myoelectricity indicated by output of a myoelectric sensor;

FIG. 10 is a table illustrating first, second, and third patterns as examples of output produced in the absence of bruxism;

FIG. 11 is a table illustrating a data structure example of sensing data;

FIG. 12 is a flowchart illustrating an example of a process related to operation of the detection device;

FIG. 13 is a flowchart illustrating an example of a process related to operation of the detection device, which is different from that of FIG. 12; and

FIG. 14 is a flowchart illustrating an example of a process related to operation of the detection device, which is different from those of FIG. 12 and FIG. 13.

### DETAILED DESCRIPTION

Embodiments of the present disclosure will be described below with reference to the drawings. The disclosure is only an example, and any modification that can be easily conceived by a person skilled in the art without departing from the spirit of the invention should be included in the scope of the present disclosure. In the drawings, the width, thickness, shape, and the like of each part are schematically illustrated compared with the actual manner, for the sake of clarity of explanation, but these are only by way of example and are not intended to limit the interpretation of the present disclosure. In the present description and drawings, elements similar to those illustrated in the previous drawings may be denoted with the same signs and a detailed description thereof may be omitted as appropriate.

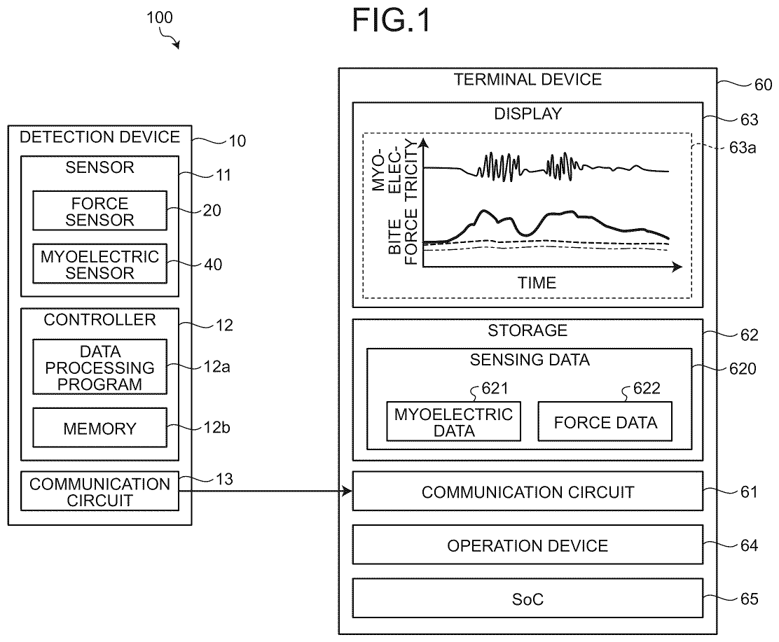

FIG. 1 is a block diagram illustrating a main configuration of a detection system 100 having a detection device 10. The detection system 100 includes the detection device 10 and a terminal device 60.

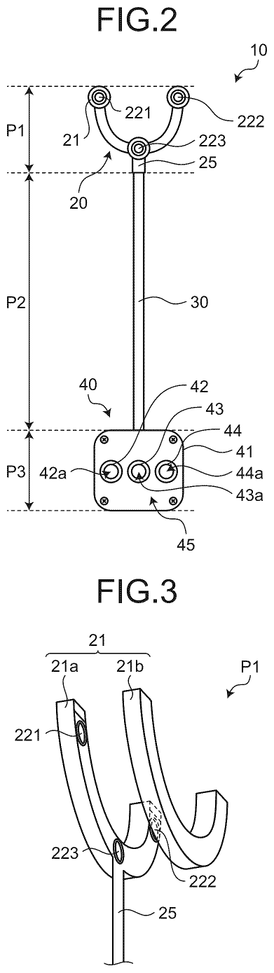

FIG. 2 is a diagram illustrating an exemplary outer shape of the detection device 10. As illustrated in FIG. 2, the detection device 10 has a first section P1, a second section P2, and a third section P3. The first section P1 includes a mouthpiece 21, a first sensor element 221, a second sensor element 222, a third sensor element 223, an FPC 25, etc. A more detailed configuration of the first section P1 will be described later in a description pertaining to a force sensor 20. The second section P2 includes an FPC 30. The third section P3 includes a casing 41, a first electrode 42, a second electrode 43, and a third electrode 44. The material of the first electrode 42, the second electrode 43, and the third electrode 44 is, for example, silver-silver chloride (Ag—AgCl) but may be gold, platinum, silver, carbon, etc.

The detection device 10 illustrated in FIG. 1 includes a sensor 11, a controller 12, and a communication circuit 13. The sensor 11 includes a force sensor 20 and a myoelectric sensor 40. The force sensor 20 and the myoelectric sensor 40 will be described sequentially below. First, the force sensor 20 will be described with reference to FIG. 3 to FIG. 7.

FIG. 3 is a schematic exploded perspective view of a main part configuration example of the first section P1. The mouthpiece 21 is formed by bonding a first part 21a and a second part 21b together. The first sensor element 221, the second sensor element 222, the third sensor element 223, and the FPC 25 are sandwiched between the first part 21a and the second part 21b.

The first sensor element 221, the second sensor element 222, and the third sensor element 223 are configured to produce an electrical effect corresponding to force. To take a specific example, the first sensor element 221, the second sensor element 222, and the third sensor element 223 are piezoelectric sensors. To take a more specific example, for example, thin-plate ceramic elements that generate a piezoelectric effect are disposed as the first sensor element 221, the second sensor element 222, and the third sensor element 223. The first sensor element 221, the second sensor element 222, and the third sensor element 223 each individually generate an electric charge corresponding to the force applied thereto.

The FPC 25 is a substrate in the form of a flexible thin film and has wiring. To take a specific example, the FPC 25 is, for example, a flexible printed circuit (FPC) board but may have another configuration that functions similarly. The first sensor element 221, the second sensor element 222, and the third sensor element 223 are wiring lines formed on the FPC 25 and are coupled to individual wiring lines.

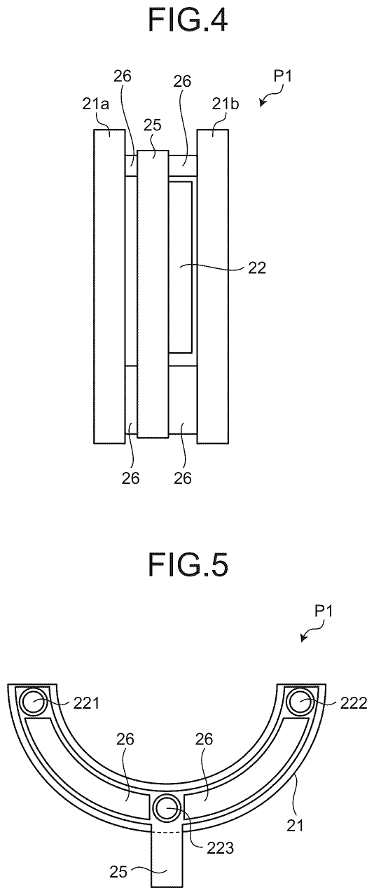

FIG. 4 is a schematic side view illustrating a mechanism of the first part 21a and the second part 21b bonded together. A sensor element 22 illustrated in FIG. 4 is any one of the first sensor element 221, the second sensor element 222, and the third sensor element 223. Hereafter, the expression “sensor element 22” refers to the first sensor element 221, the second sensor element 222, and the third sensor element 223 collectively.

As illustrated in FIG. 4, the FPC 25 is bonded to the first part 21a on one side and to the second part 21b on the other side with adhesive 26 interposed therebetween. The sensor element 22 is provided on the other side of the FPC 25 in the example illustrated in FIG. 4, but may be provided on the one side of the FPC 25.

The mouthpiece 21 is a mouthpiece adapted to a curved shape of a bifurcated portion of the FPC 25 where the first sensor element 221, the second sensor element 222, and the third sensor element 223 are provided. As illustrated in FIG. 4, the FPC 25 is adhesively fixed such that a gap is formed between the FPC 25 and the mouthpiece 21, inside the mouthpiece 21.

The mouthpiece 21 is formed, for example, but not limited to, using a resin (for example, resin or silicone) having a certain degree of flexibility. The specific composition of the mouthpiece 21 can be changed as appropriate as long as force can be transmitted to the first sensor element 221, the second sensor element 222, and the third sensor element 223. It is preferable that the mouthpiece 21 is produced by molding a dental impression acquired from each wearer.

FIG. 5 is a schematic plan view of a main part configuration example of the first section P1. As illustrated in FIGS. 2, 3, 5, and 7, the FPC 25 has a bifurcated shape at one end side having the first sensor element 221 and the second sensor element 222. The first sensor element 221 and the second sensor element 222 are provided near distal ends of the first section P1. The FPC 25 also has a strip-like shape at a proximal end side of the first section P1 that is continuously connected with the FPC 30 of the second section P2. The third sensor element 223 is provided near a connection position between the bifurcated portion and the strip portion.

As illustrated in FIG. 5, the adhesive 26 is mostly applied to, for example, but not limited to, a region between the third sensor element 223 and the first sensor element 221 and a region between the third sensor element 223 and the second sensor element 222 in the bifurcated portion. However, the adhesive may be applied in any way that can maintain the integration of the mouthpiece 21 and the FPC 25.

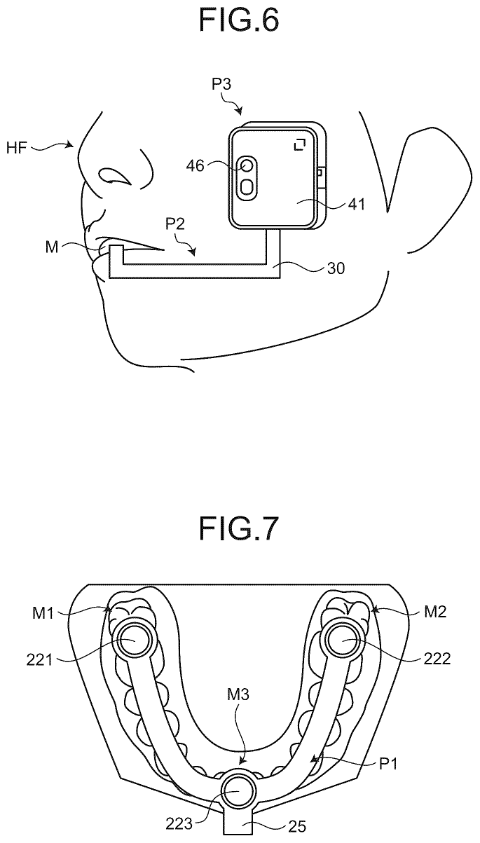

FIG. 6 is a schematic diagram illustrating an example of attachment of the detection device 10 to a human. As illustrated in FIG. 6, when the detection device 10 is attached to a human, the casing 41 of the third section P3 is attached such that a surface exposing the first electrode 42, the second electrode 43, and the third electrode 44 is in close proximity to or contact with an outer side area of the cheek of the human HF. Specifically, for example, conductive gels 42a, 43a, and 44a are provided on the first electrode 42, the second electrode 43, and the third electrode 44 of the casing 41, respectively, and double-sided tape 45 for skin is provided on the remaining portion of the casing 41 to allow the casing 41 to be attached to the human HF. The material of the double-sided tape is preferably silicone.

In the embodiment, the double-sided tape 45 for skin is applied on the surface of the casing 41 on which the first electrode 42, the second electrode 43, and the third electrode 44 are provided. The double-sided tape 45 for skin is applied throughout the surface of the casing 41 in the area surrounding the first electrode 42, the second electrode 43, and the third electrode 44. The conductive gels 42a, 43a, and 44a cover respective outer electrode surfaces of the first electrode 42, the second electrode 43, and the third electrode 44. When the detection device 10 is attached to a human, the first section P1 is placed in the mouth M of the human. The FPC 30 of the second section P2 extends having a length by which the first section P1 can be coupled to the casing 41 when the detection device 10 is attached to the human.

The FPC 30, which is provided in the second section P2, is an FPC continuous from the proximal end side of the first section P1 of the FPC 25. The FPC 30 serves as wiring that couples the first sensor element 221, the second sensor element 222, and the third sensor element 223 to a circuit in the casing 41 of the third section P3. A circuit or the like is provided in the casing 41 to convert outputs of the first sensor element 221, the second sensor element 222, and the third sensor element 223 into digital data that can be handled individually. The circuit or the like is included in the configuration of the force sensor 20 in the embodiment.

FIG. 7 is a schematic diagram illustrating the arrangement of the first section P1 in the mouth M illustrated in FIG. 6. As illustrated in FIG. 7, the first section P1 is held in the mouth of the human such that the first sensor element 221 is located near one rearmost molar M1 among the teeth in the mouth M, the second sensor element 222 is located near the other rearmost molar M2 among the teeth in the mouth M, and the third sensor element 223 is located near incisors M3 closest to the center among the teeth in the mouth M. In other words, the shape and the extension length of the bifurcated mouthpiece 21 and the FPC 25 as well as the arrangement of the first sensor element 221, the second sensor element 222, and the third sensor element 223 in the first section P1 are predetermined such that the above positional relation between the molars M1, M2, and incisors M3 and the first, second, and third sensor elements 221, 222, and 223 is satisfied. Since there can be individual differences in the size of the human mouth M and the positions of the molars M1, M2 and incisors M3 in the mouth M, the first section P1 may be customized for each human, or a design of the first section P1 based on a relation with a typical human adult dental model may be adopted as a standard design. The first section P1 is for the upper jaw and is attached to teeth in the upper jaw. However, the first section P1 may be for the lower jaw and attached to teeth in the lower jaw. Strictly speaking, whether the first section P1 is for the upper jaw or for the lower jaw is determined for each individual human, based on the dentist's judgment. In this way, the mouthpiece 21 may be attached to either the upper jaw or the lower jaw inside the human mouth M.

When the upper and lower teeth of the jaws move to make contact at the position of the molar M1, the force caused by the contact is detected by the first sensor element 221. When the upper and lower teeth of the jaws move to make contact at the position of the molar M2, the force caused by the contact is detected by the second sensor element 222. When the upper and lower teeth of the jaws move to make contact at the position of the incisors M3, the force caused by the contact is detected by the third sensor element 223. In the embodiment, therefore, the first sensor element 221, the second sensor element 222, and the third sensor element 223 function as the force sensor 20 in the human mouth.

Either the shape of the mouthpiece 21 and the FPC 25 illustrated in FIGS. 2 and 7 or the shape of the mouthpiece 21 and the FPC 25 illustrated in FIGS. 3 to 5 may be employed. The shapes of the components of the first section P1 illustrated in FIGS. 3 to 5 are schematic shapes to illustrate the relative positional relation between the components of the first section P1 and do not represent a specific manner. The specific configuration of the first section P1 is not limited to the configuration illustrated in FIGS. 2 and 7 as long as the mouthpiece allows a force sensor to be disposed to detect force corresponding to the bite of the teeth in the human mouth. Although a case where three sensor elements 22 function as the force sensor 20 is illustrated here, a force sensor corresponding to each of teeth may be disposed. For example, if the upper jaw has 14 teeth, 14 force sensors may be disposed.

The following describes the myoelectric sensor 40 illustrated in FIG. 1 with reference to FIGS. 1, 2, 6, and 8.

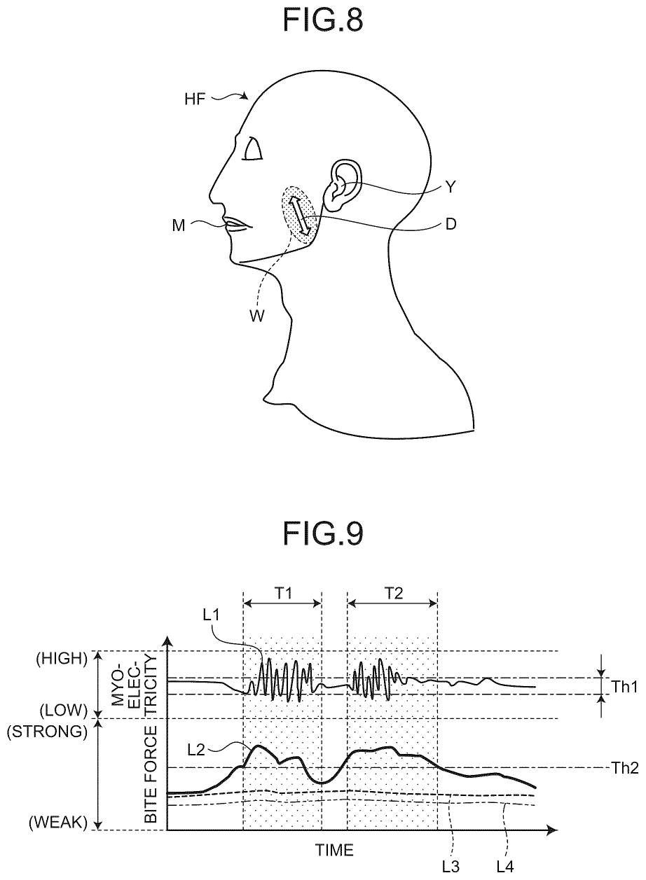

FIG. 8 is a schematic diagram illustrating an attachment position of the first electrode 42, the second electrode 43, and the third electrode 44. The casing 41 of the third section P3 is attached to the human HF such that the first electrode 42, the second electrode 43, and the third electrode 44 are aligned along a direction D in a region W illustrated in FIG. 8. The region W is a region closer to the ear Y in the human cheek area between the mouth M and the ear Y of the human HF. The direction D is, for example, a direction generally orthogonal to a straight line connecting the ear Y and the mouth M, and substantially coincides with a straight line from around the corner of one of the two eyes to the neck in the human HF. For example, as described with reference to FIG. 6 above, the casing 41 of the third section P3 is attached to the human HF such that the surface exposing the first electrode 42, the second electrode 43, and the third electrode 44 is in close proximity to or contact with the cheek area of the human HF, whereby the first electrode 42, the second electrode 43, and the third electrode 44 are aligned along the direction D in the region W.

The feature of the region W and the direction D described above is only a typical example and in practice may be adjusted for each individual human. It is preferable that the adjustment is made assuming that the positions of the first electrode 42, the second electrode 43, and the third electrode 44 in the third section P3 overlap a superficial part (superficial layer) of the human masseter muscle.

The myoelectric sensor 40 illustrated in FIG. 1 includes a function as a differential amplifier using the first electrode 42, the second electrode 43, and the third electrode 44. One of the first electrode 42, the second electrode 43, and the third electrode 44 is a reference electrode (REF). One of the first electrode 42, the second electrode 43, and the third electrode 44 that is not a reference electrode is an exploring electrode. One of the first electrode 42, the second electrode 43, and the third electrode 44 that is neither a reference electrode nor an exploring electrode is a ground electrode (GND). The ground electrode assumes a ground potential (0 V). Ideally, it is preferable that the reference electrode assumes the same potential (0 V) as the ground potential, but in practice, the reference electrode assumes a potential subjected to noise components (noise) at the position where the third section P3 is disposed. The exploring electrode is disposed in the region W to capture myoelectricity and assumes a potential corresponding to the myoelectricity. The myoelectricity refers to an electrical signal generated in the muscle corresponding to the superficial part (superficial layer) of the human masseter muscle described above.

In practice, the potential of the exploring electrode may include the noise described above. In the embodiment, therefore, similar noise is detected at the reference electrode so that a potential corresponding to myoelectricity can be detected more accurately based on the difference between the potential of the exploring electrode and the potential of the reference electrode. More specifically, an electrical signal corresponding to the difference between the potential of the exploring electrode and the potential of the ground electrode is input to one of two inputs of an amplifier including an operational amplifier or the like. An electrical signal corresponding to the difference between the potential of the reference electrode and the potential of the ground electrode is input to the other of two inputs of the amplifier. As a result, electrical signals in phase of the two inputs of the amplifier cancel each other, whereas signals different from each other, such as signals of opposite phases, are amplified. The myoelectric sensor 40 in the embodiment therefore can capture myoelectricity more accurately.

A circuit or the like is provided in the casing 41 to convert an output of the myoelectric sensor 40 serving as the amplifier described above into digital data. The circuit or the like is included in the configuration of the myoelectric sensor 40 in the embodiment.

The controller 12 illustrated in FIG. 1 performs processing based on a signal output from the sensor 11. The following describes the processing performed by the controller 12 with reference to FIG. 1 and FIGS. 9 to 11.

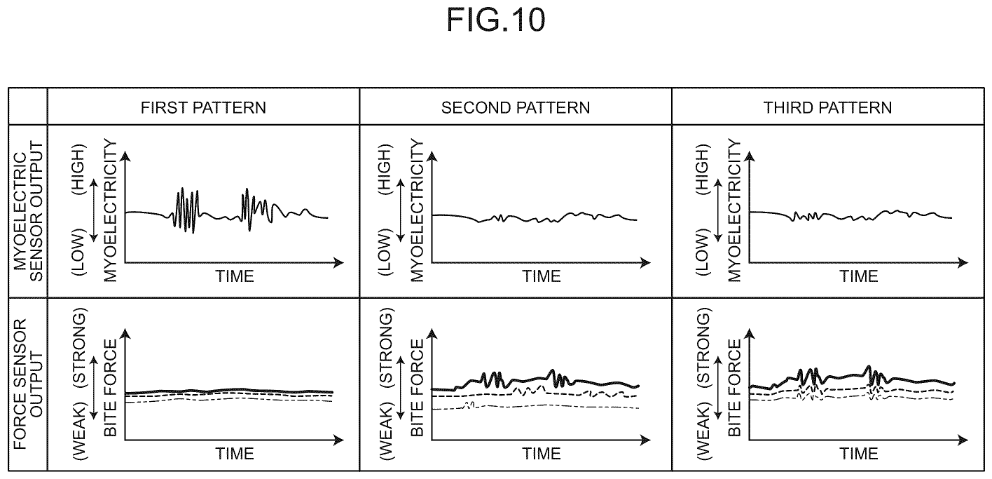

FIG. 9 is a graph illustrating an example of the relation in time sequence between human bite force indicated by output of the force sensor 20 and myoelectricity indicated by output of the myoelectric sensor 40. Among four lines in the graph illustrated in FIG. 9, lower three lines L2, L3, and L4 represent the strength of output of the force sensor 20, that is, the strength of the human bite force. The lower three lines L2, L3, and L4 represent individual outputs of the first sensor element 221, the second sensor element 222, and the third sensor element 223, respectively. Line L2 represents the output of the first sensor element 221, line L3 represents the output of the second sensor element 222, and line L4 represents the output of the third sensor element 223. Among the four lines, the upper one line L1 represents the strength of output of the myoelectric sensor 40, that is, the strength of myoelectricity. In FIG. 9, a dashed line between the lower three lines L2, L3, L4 and the upper one line L1 separates the graph representing bite force from the graph representing myoelectricity. The vertical axis direction of the graphs in FIG. 9 and FIG. 10 described later indicates the strength of output. The horizontal axis direction of the graphs in FIGS. 9 and 10 indicates the passage of time.

During periods T1 and T2 in the graph illustrated in FIG. 9, one (line L2) of the lower three lines L2, L3, and L4, which represent the strength of bite force, represents a significantly strong bite force, compared with the other two (lines L3 and L4). This indicates occurrence of a situation in which the upper and lower teeth of the jaws are in close proximity to contact each other to apply pressing force to the sensor element 22 at a position of one of molar M1, molar M2, and incisors M3. Assuming that line L2 represents output of the first sensor element 221, the situation occurs where the upper and lower teeth of the jaws are so close that they are in contact with each other at the molar M1 to apply pressing force to the sensor element 22. Such a situation can occur when bruxism is occurring. The upper one line L1 representing the strength of myoelectricity also has significantly large fluctuations of output during periods T1 and T2, compared with the other periods. This suggests movement of the masseter muscle, that is, force acting in a direction of pulling the lower jaw toward the upper jaw. This serves as a more reliably suggestion supporting the occurrence of bruxism.

As described above, in the embodiment, the occurrence of bruxism can be determined based on data in which the output of the force sensor 20 and the output of the myoelectric sensor 40 are synchronized in time. In contrast, with only one of the output of the force sensor 20 and the output of the myoelectric sensor 40, it is difficult to accurately determine the occurrence of bruxism. The following describes a reference example in which false detection is caused with only one of the output of the force sensor 20 and the output of the myoelectric sensor 40 with reference to FIG. 10.

FIG. 10 is a table illustrating first, second, and third patterns as examples of output produced in the absence of bruxism. The first pattern is a pattern in a case where the human eyelids are tightly closed. The second pattern is a pattern in a case where the human tongue touches the teeth in the mouth. The third pattern is a pattern in a case where the human lips touch the teeth. The “myoelectric sensor output” in FIG. 10 indicates the strength of output of a configuration similar to the myoelectric sensor 40. The “force sensor output” in FIG. 10 indicates output of a configuration similar to the force sensor 20 having the first sensor element 221, the second sensor element 222, and the third sensor element 223. In the reference example, either “myoelectric sensor output” or “force sensor output” can be obtained.

As indicated by “First Pattern” in FIG. 10, the “myoelectric sensor output” has significantly large fluctuations of output even when the human eyelids are tightly closed, in the same manner as when bruxism occurs. In the reference example in which only “myoelectric sensor output” is obtained, therefore, it is difficult to distinguish between a case where bruxism occurs and a case where the human eyelids are tightly closed.

As indicated by “Second Pattern” and “Third Pattern” in FIG. 10, the output of the force sensor 20 significantly fluctuates even when the tongue or lips touch the teeth. In the reference example in which only “force sensor output” is obtained, therefore, it is difficult to distinguish between a case where bruxism occurs and a case where the tongue or lips touch the teeth.

In contrast, in the embodiment, the output of the force sensor 20 does not significantly fluctuate even when a situation similar to “First Pattern” occurs. As a result, in the embodiment, it is possible to distinguish between a case where bruxism occurs and a case where the human eyelids are tightly closed. In the embodiment, the output of the myoelectric sensor 40 does not significantly fluctuate even when a situation similar to “Second Pattern” or “Third Pattern” occurs. As a result, in the embodiment, it is possible to distinguish between a case where bruxism occurs and a case where the tongue or lips touch the teeth. The embodiment therefore can more accurately distinguish between bruxism and an event other than bruxism.

As indicated in periods T1 and T2 in FIG. 9, synchronization in time between the output of the force sensor 20 and the output of the myoelectric sensor 40 is important for distinguishing bruxism from the others. In the embodiment, the controller 12 of the detection device 10 illustrated in FIG. 1 generates data in which the output of the force sensor 20 and the output of the myoelectric sensor 40 are synchronized in time.

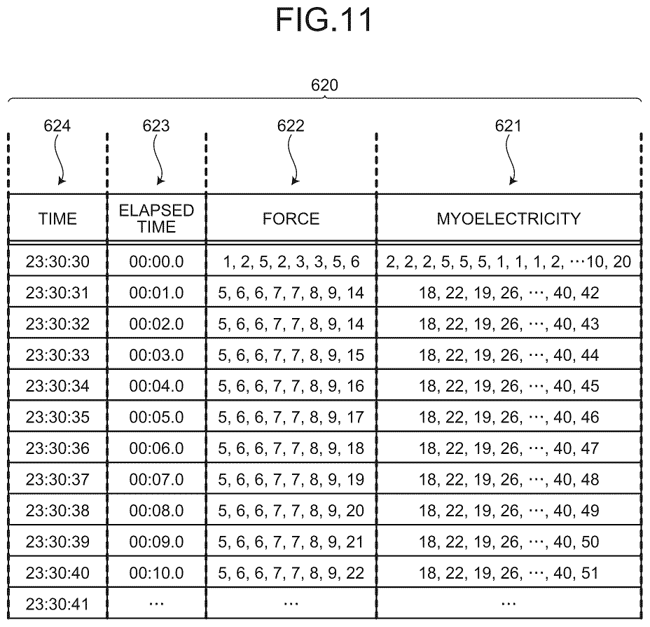

FIG. 11 is a table illustrating a data structure example of sensing data 620. The sensing data 620 includes myoelectric data 621, force data 622, synchronization data 623, and time data 624. The myoelectric data 621 is data indicating the output of the myoelectric sensor 40. The force data 622 is data indicating the output of the force sensor 20. The synchronization data 623 is data indicating the elapsed time since the start of sensing by the detection device 10. The controller 12 generates a record with a new parameter of the synchronization data 623 every time a unit time passes after the start of sensing. The controller 12 associates data of the force sensor 20 and data of the myoelectric sensor 40 produced during the unit time with the parameter of the synchronization data 623. In the example illustrated in FIG. 11, the unit time is one second, but the unit time is not limited to this and can be changed as appropriate.

In the embodiment, the force sensor 20 produces an output at 8 cycles per second (8 Hz), and the myoelectric sensor 40 produces an output at 512 cycles per second (512 Hz). The controller 12 acquires the output of the force sensor 20 and the output of the myoelectric sensor 40 and generates data that can be handled on a unit time basis. Each record in the table illustrated in FIG. 11 represents the data for each unit time thus generated by the controller 12 in a human-interpretable form and is not the actual data structure itself. The values of the myoelectric data 621 and the values of the force data 622 illustrated in FIG. 11 are only examples and are not intended to limit the output form of the myoelectric data 621 and the output form of the force data 622. The output cycle of the force sensor 20 and the output cycle of the myoelectric sensor 40 are only examples and not limited to these, and can be changed as appropriate.

The time during which data is obtained within a series of time periods of sensing by the force sensor 20 and the myoelectric sensor 40, can be identified by a parameter of the synchronization data 623. For example, a record with a parameter of “00:00.0” of the synchronization data 623 is data indicating the output of the force sensor 20 and the output of the myoelectric sensor 40 obtained during a period from immediately after the start of sensing until one second has passed. A record with a parameter “00:01.0” of the synchronization data 623 is data indicating the output of the force sensor 20 and the output of the myoelectric sensor 40 obtained during a period from the time when one second has elapsed since the start of sensing until two seconds have elapsed. Other records with parameters of the synchronization data 623 can be interpreted similarly.

The sensing data 620 illustrated in FIG. 11 further includes the time data 624 in addition to the myoelectric data 621, the force data 622, and the synchronization data 623. The time data 624 indicates the time when the sensing is performed. In other words, the parameter of the time data 624 added to each record indicates the time that matches the point in time of the synchronization data 623. The processing of adding the time data 624 to data may be performed by the controller 12, but in the embodiment, the processing is performed by an SoC 65 of the terminal device 60.

As illustrated in FIG. 1, the controller 12 includes a data processing program 12a and a memory 12b. The data processing program 12a is a software program for generating data that can be handled on a unit time basis, based on the output of the force sensor 20 and the output of the myoelectric sensor 40. The configuration of the controller 12 includes an arithmetic circuit for reading and executing the data processing program 12a. The memory 12b functions as a storage region capable of temporarily storing data representing the output of the force sensor 20 and the output of the myoelectric sensor 40 as well as data generated using the data processing program 12a. The controller 12 may be a circuit that implements the function as the data processing program 12a in hardware and has a storage region to function as the memory 12b.

The communication circuit 13 illustrated in FIG. 1 performs processing related to communication with an external apparatus such as the terminal device 60. Specifically, the communication circuit 13 has a circuit or the like to function as a network interface controller (NIC). The communication circuit 13 communicates with an external apparatus using predetermined communication protocols. The predetermined communication protocols may be a known communication protocol such as the Internet protocol and various protocols for implementing the communication protocol, or may be a dedicated protocol defined between the communication circuit 13 and the terminal device 60. The form of the communication line between the communication circuit 13 and the terminal device 60 may be wired, wireless, or a combination of wired and wireless, and may include a public communication line in part.

The controller 12 in the embodiment, for example, acquires the output from the force sensor 20 and the output from the myoelectric sensor 40, adds a parameter of the synchronization data 623 for each unit time to make a package, and transmits the package to the terminal device 60 via the communication circuit 13.

In the embodiment, the controller 12 and the communication circuit 13 are provided in the casing 41 (see FIGS. 2 and 6), but the specific manner for implementing the controller 12 and the communication circuit 13 in the detection device 10 is not limited as long as their functions can be achieved.

A battery or the like for supplying and storing power necessary for the operation of the sensor 11, the controller 12, and the communication circuit 13 may be provided in the casing 41. An interface or the like that allows a power line for supplying the power to be coupled to the casing 41 may be further provided in the casing 41.

The following describes the terminal device 60 illustrated in FIG. 1. The terminal device 60 is, for example, a portable device such as a smartphone, but not limited to this and may be an information processing device having other forms, such as a stationary information processing device such as a personal computer. The terminal device 60 includes a communication circuit 61, a storage 62, a display 63, an operation device 64, and an SoC 65.

The communication circuit 61 performs processing related to communication with an external apparatus such as the communication circuit 13. The communication circuit 61 has a circuit or the like to function as an NIC, in the same manner as the communication circuit 13. In the embodiment, a communication protocol employed by the terminal device 60 and a communication protocol employed by the communication circuit 13 are selected so that communication is established between the terminal device 60 and the communication circuit 13.

The storage 62 stores the sensing data 620 described with reference to FIG. 11. Specifically, the storage 62 has, for example, a storage circuit such as a flash memory provided in the terminal device 60. As described with reference to FIG. 11, the sensing data 620 includes the myoelectric data 621 and the force data 622. Although not illustrated in FIG. 1, the sensing data 620 in the embodiment further includes the synchronization data 623 and the time data 624.

The display 63 performs display output in accordance with the processing performed in the terminal device 60. The display 63 has, for example, a display device such as an organic electroluminescence (EL) display or a liquid crystal display and performs display output in accordance with the processing performed by the SoC 65. The display 63 in the embodiment performs display output in accordance with the contents of the sensing data 620, for example, as illustrated in a graph 63a in FIG. 1. The graph 63a illustrated in FIG. 1 is similar to, for example, but not limited to, the graph illustrated in FIG. 9 and reflects the actual outputs of the force sensor 20 and the myoelectric sensor 40.

The graph 63a is visualized, for example, by plotting a plurality of records contained in the sensing data 620 illustrated in FIG. 11 in time sequence.

The operation device 64 receives inputs to the terminal device 60 from the user of the terminal device 60. The operation device 64 is, for example, a touch panel integrated with the display 63 but not limited to this and may be an input device that employs any other input method.

The SoC 65 performs information processing performed in the terminal device 60. The SoC 65 has a configuration (system on a chip (SoC)) in which multiple functions are implemented in a single integrated circuit, but may include a plurality of circuits that function similarly. The SoC 65 receives data transmitted from the detection device 10 via, for example, the communication circuit 61. The SoC 65 adds the time data 624 to the data received from the detection device 10 and stores the data in the storage 62 as sensing data 620. The SoC 65 causes the display 63 to perform display output in accordance with the user's input operation via the operation device 64. Specific forms of the display output include, for example, the graph 63a.

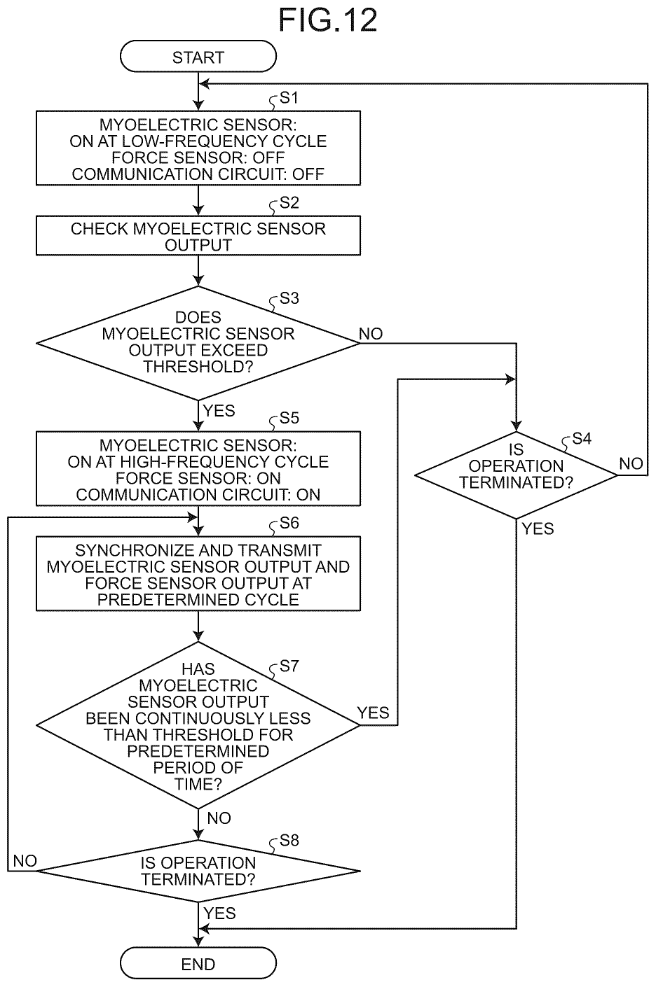

The following describes the process related to the operation of the detection device 10 worn as illustrated in FIG. 6, with reference to FIGS. 12 to 14. The processing described with reference to FIGS. 12 to 14 is implemented by the controller 12 executing the data processing program 12a. In the flowcharts in FIGS. 12 to 14, the operation of the configuration is denoted as “ON” and the non-operation is denoted as “OFF”.

FIG. 12 is a flowchart illustrating an example of a process related to the operation of the detection device 10. After the operation starts, the controller 12 causes the myoelectric sensor 40 to operate at low-frequency cycles (step S1). The controller 12 causes the force sensor 20 and the communication circuit 13 not to operate in the processing at step S1.

The operation at low-frequency cycles in the description with reference to FIGS. 12 to 14 refers to, for example, the operation of performing sensing and producing an output at a time cycle that is 10 times the above unit time. The relation between the low-frequency cycle and the multiplier of the unit time is not limited to this and can be changed as appropriate. However, in the operation at low-frequency cycles, the time interval in sensing is longer than in the operation at high-frequency cycles described below.

The controller 12 checks the output of the myoelectric sensor 40 caused to operate at low-frequency cycles by the processing at step S1 (step S2). The controller 12 determines whether the output of the myoelectric sensor 40 exceeds a threshold by checking the output of the myoelectric sensor 40 in the processing at step S2 (step S3). If it is determined that the output of the myoelectric sensor 40 does not exceed the threshold (No at step S3), the process moves to step S1 unless the operation of the detection device 10 is terminated (No at step S4). In other words, the operating state of the myoelectric sensor 40 at low-frequency cycles and the non-operating state of the force sensor 20 and the communication circuit 13 continue. A condition for the end of operation in the processing at step S4 is, for example, when the user who is the wearer of the detection device 10 performs a terminate operation of the detection device 10 with a terminal device application executed on the terminal device 60. More specifically, it is assumed that the user performs an operation to start the terminal device application before going to sleep and then terminates the terminal device application after the user wakes up. The terminal device application may be automatically terminated after a preset period of time has elapsed (for example, after 10 hours) since the startup of the terminal device application. The condition for ending operation may also be applied to the processing at step S8, the processing at step S14, the processing at step S24, and the processing at step S28 described later.

The threshold in the processing at step S3 and the processing at step S7 described later is, for example, a first threshold Th1 illustrated in FIG. 9. If an output that exceeds the range of the first threshold Th1 is produced, it is determined that the output of the myoelectric sensor 40 exceeds the threshold. If an output that falls within the range of the first threshold Th1 is produced, it is determined that the output of the myoelectric sensor 40 is less than the threshold. An output that overlaps with either one of lines that define the range of the first threshold Th1 may be considered as being neither larger nor less than the threshold, or may be determined to be larger or less than the threshold.

On the other hand, if it is determined that the output of the myoelectric sensor 40 exceeds the predetermined threshold at step S3 (Yes at step S3), the controller 12 causes the myoelectric sensor 40 to operate at high-frequency cycles (step S5). The controller 12 also causes the force sensor 20 and the communication circuit 13 to operate in the processing at step S5. The operation cycles of the force sensor 20 in the processing at step S5 are high-frequency cycles, similar to those of the myoelectric sensor 40.

The operation at high-frequency cycles in the description with reference to FIGS. 12 to 14 refers to, for example, the operation of producing output of sensing at a cycle of the unit time described above. In the embodiment, sensing for one second is performed correspondingly to an output, regardless of whether the sensing is performed at low-frequency cycles or at high-frequency cycles. However, the length of the period in which the sensing is performed in each cycle can be changed as appropriate.

The controller 12 synchronizes the outputs of the myoelectric sensor 40 and the force sensor 20 operating at high-frequency cycles and transmits the outputs to the terminal device 60 at a predetermined cycle (step S6). The predetermined cycle is, for example, a cycle similar to the high-frequency cycle (every second in the embodiment) but not limited to this and can be changed as appropriate. The “synchronization” in the description with reference to FIGS. 12 to 14 refers to, for example, the processing of adding the synchronization data 623 to the myoelectric data 621 and the force data 622 and generating data corresponding to a record on a one second basis described with reference to FIG. 11. The data generated in this way is transmitted via the communication circuit 13.

The controller 12 determines whether the output of the myoelectric sensor 40 has been continuously less than the threshold for a predetermined period of time (step S7). If it is determined that the output of the myoelectric sensor 40 has not been continuously less than the threshold for a predetermined period of time (No at step S7), the process moves to step S6 unless the operation of the detection device 10 is terminated (No at step S8). In other words, the operating state of the force sensor 20 and the myoelectric sensor 40 at high-frequency cycles, the generation of data, and the transmission of data via the communication circuit 13 continue.

The predetermined period of time in the description with reference to FIGS. 12 to 14 is, for example, five minutes but not limited to this and can be changed as appropriate. However, it is preferred that the predetermined period of time is significantly longer than the unit time of the high-frequency cycle.

On the other hand, if it is determined that the output of the myoelectric sensor 40 has been continuously less than a threshold for the predetermined period of time at step S7 (Yes at step S7), the process moves to step S4. If the operation of the detection device 10 has not been terminated (No at step S4), the process moves to step S1. In other words, the detection device 10 makes a transition to the operating state of the myoelectric sensor 40 at low-frequency cycles and a transition to the non-operating state of the force sensor 20 and the communication circuit 13.

If the operation of the detection device 10 is terminated at step S4 (Yes at step S4) and the operation of the detection device 10 is terminated at step S8 (Yes at step S8), the processing by the controller 12 ends.

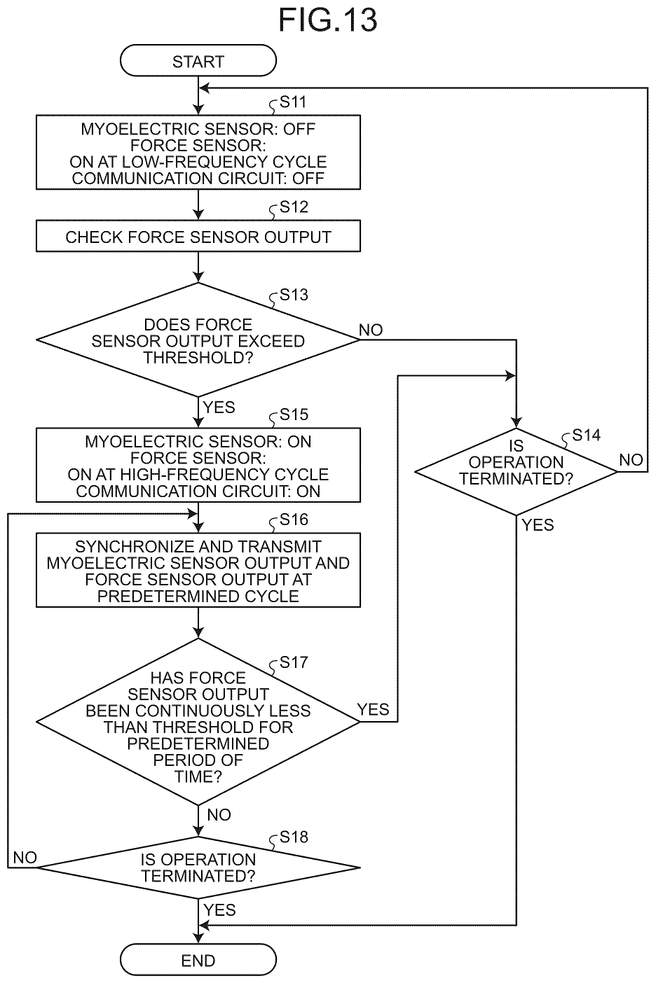

FIG. 13 is a flowchart illustrating an example of a process related to operation of the detection device 10, which is different from that of FIG. 12. After the operation starts, the controller 12 causes the force sensor 20 to operate at low-frequency cycles (step S11). The controller 12 causes the myoelectric sensor 40 and the communication circuit 13 not to operate in the processing at step S11.

The controller 12 checks the output of the force sensor 20 caused to operate at low-frequency cycles by the processing at step S11 (step S12). The controller 12 determines whether the output of the force sensor 20 exceeds a threshold by checking the output of the force sensor 20 in the processing at step S12 (step S13). If it is determined that the output of the force sensor 20 does not exceed the threshold (No at step S13), the process moves to step S11 unless the operation of the detection device 10 is terminated (No at step S14). In other words, the operating state of the force sensor 20 at low-frequency cycles and the non-operating state of the myoelectric sensor 40 and the communication circuit 13 continue.

The threshold in the processing at step S13 and the processing at step S17 described later is, for example, a second threshold Th2 illustrated in FIG. 9. If an output higher than the second threshold Th2 is produced, it is determined that the output of the force sensor 20 exceeds the threshold. If an output that stays below the second threshold Th2 is produced, it is determined that the output of the force sensor 20 is less than the threshold. An output that overlaps with a line that defines the second threshold Th2 may be considered as being neither larger nor less than the threshold, or may be considered as being larger or less than the threshold.

On the other hand, if it is determined that the output of the force sensor 20 exceeds the predetermined threshold at step S13 (Yes at step S13), the controller 12 causes the force sensor 20 to operate at high-frequency cycles (step S15). The controller 12 also causes the myoelectric sensor 40 and the communication circuit 13 to operate in the processing at step S15. The operation cycles of the myoelectric sensor 40 in the processing at step S15 are high-frequency cycles, similar to those of the force sensor 20.

The controller 12 synchronizes the outputs of the myoelectric sensor 40 and the force sensor 20 operating at high-frequency cycles and transmits the outputs to the terminal device 60 at a predetermined cycle (step S16). The controller 12 determines whether the output of the force sensor 20 has been continuously less than the threshold for a predetermined time (step S17). If it is determined that the output of the force sensor 20 has not been continuously less than the threshold for a predetermined period of time (No at step S17), the process moves to step S16 unless the operation of the detection device 10 is terminated (No at step S18). In other words, the operating state of the force sensor 20 and the myoelectric sensor 40 at high-frequency cycles, the generation of data, and the transmission of data via the communication circuit 13 continue.

On the other hand, if it is determined that the output of the force sensor 20 has been continuously less than a threshold for the predetermined period of time at step S17 (Yes at step S17), the process moves to step S14. If the operation of the detection device 10 has not been terminated (No at step S14), the process moves to step S11. In other words, the detection device 10 makes a transition to the operating state of the force sensor 20 at low-frequency cycles and a transition to the non-operating state of the myoelectric sensor 40 and the communication circuit 13.

If the operation of the detection device 10 is terminated at step S14 (Yes at step S14) and the operation of the detection device 10 is terminated at step S18 (Yes at step S18), the processing by the controller 12 ends.

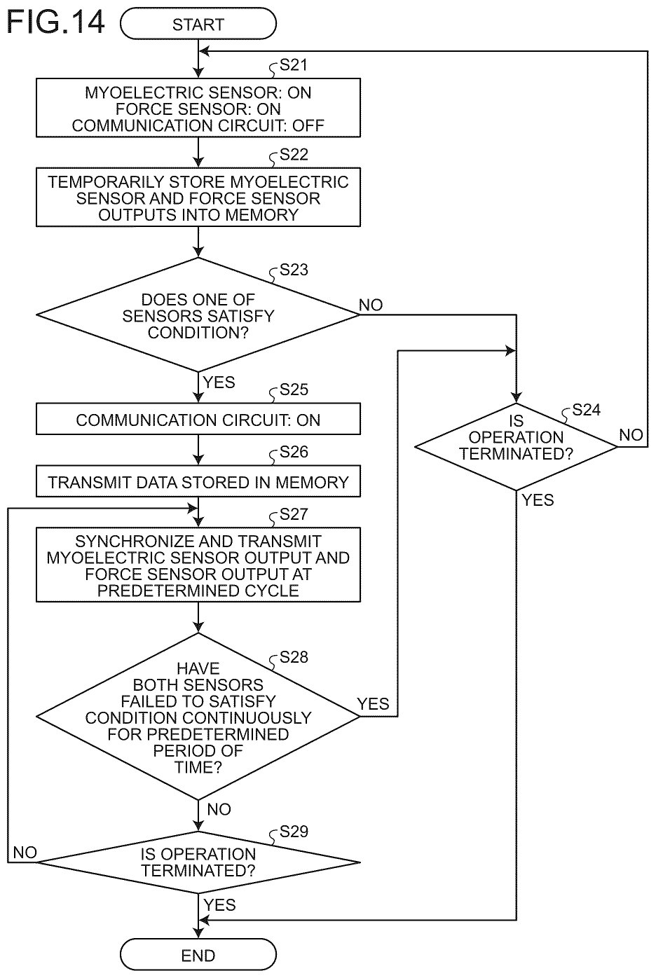

FIG. 14 is a flowchart illustrating an example of a process related to operation of the detection device 10, which is different from those of FIG. 12 and FIG. 13. After the operation starts, the controller 12 causes the force sensor 20 and the myoelectric sensor 40 to operate (step S21). The controller 12 causes the communication circuit 13 not to operate in the processing at step S21.

The operation cycle of each of the force sensor 20 and the myoelectric sensor 40 in the processing at step S21 may be a low-frequency cycle or a high-frequency cycle.

The controller 12 generates data in which the outputs of the force sensor 20 and the myoelectric sensor 40 caused to operate by the processing at step S21 are synchronized, and temporarily stores the data in the memory 12b (step S22). In the operation of temporarily storing the data in the memory 12b in the processing at step S22, for example, only the latest five minutes of data is retained and data after a lapse of five minutes is discarded. The length of the retention period can be changed as appropriate.

The controller 12 determines whether one of the force sensor 20 and the myoelectric sensor 40 satisfies a condition (step S23). The conditions referred to in the processing at step S23 and the processing at step S28 described later are as follows: in the case of the force sensor 20, for example, when the output of the force sensor 20 exceeds a predetermined threshold, the condition is determined to being satisfied, in the same manner as in the determination at step S13 described above; in the case of the myoelectric sensor 40, when the output of the myoelectric sensor 40 exceeds a predetermined threshold, the condition is determined to being satisfied, in the same manner as in the determination at step S3 described above.

If it is determined that neither of the sensors satisfies the condition at step S23 (No at step S23), the process moves to step S21 unless the operation of the detection device 10 is terminated (No at step S24). In other words, the operation of the force sensor 20 and the myoelectric sensor 40 and the operation of temporarily storing data in the memory 12b continue.

On the other hand, if it is determined that one of the sensors satisfies the condition at step S23 (Yes at step S23), the controller 12 causes the communication circuit 13 to operate (step S25). The controller 12 transmits the data temporarily stored in the memory 12b in the processing at step S22 to the terminal device 60 via the communication circuit 13 (step S26).

If the force sensor 20 and the myoelectric sensor 40 are operating at low-frequency cycles at step S21, the operation cycles of the force sensor 20 and the myoelectric sensor 40 become high-frequency cycles at the point in time at step S25.

The controller 12 synchronizes the outputs of the myoelectric sensor 40 and the force sensor 20 and transmits the outputs to the terminal device 60 at a predetermined cycle (step S27).

The controller 12 determines whether neither the force sensor 20 nor the myoelectric sensor 40 has satisfied the condition continuously for a predetermined period of time (step S28). If it is determined that both sensors have failed to satisfy the condition continuously for a predetermined period of time (No at step S28), the process moves to step S27 unless the operation of the detection device 10 is terminated (No at step S29). In other words, the operating state of the force sensor 20 and the myoelectric sensor 40 at high-frequency cycles, the generation of data, and the transmission of data via the communication circuit 13 continue.

On the other hand, if it is determined that neither the force sensor 20 nor the myoelectric sensor 40 has satisfied the condition continuously for a predetermined period of time at step S28 (Yes at step S28), the process moves to step S24. If the operation of the detection device 10 has not been terminated (No at step S24), the process moves to step S21. In other words, the detection device 10 transitions to a state in which the operation of the force sensor 20 and the myoelectric sensor 40 and the operation of temporarily storing data in the memory 12b are performed.

If the operation of the detection device 10 is terminated at step S24 (Yes at step S24) and the operation of the detection device 10 is terminated at step S29 (Yes at step S29), the processing by the controller 12 ends.

Three processes performed by the controller 12 have been exemplarily described with reference to FIGS. 12 to 14. The controller 12 in the embodiment operates according to any of these three processes. The process to be applied may be determined in advance or may be selectable. For example, the detection device 10 may be provided such that the process to be applied to the controller 12 can be changed through an operation on an operation device such as a switch 46 illustrated in FIG. 6. The process to be applied to the controller 12 may be changeable through an operation on the operation device 64 of the terminal device 60 and communication between the communication circuit 61 and the communication circuit 13.

As described above, according to the embodiment, the detection device 10 includes a force sensor (for example, first sensor element 221, second sensor element 222, third sensor element 223) disposed at a mouthpiece (for example, mouthpiece 21), a myoelectric sensor (for example, myoelectric sensor 40) attachable to a human cheek, and a controller (for example, controller 12) configured to perform output in which an output of the force sensor is synchronized with an output of the myoelectric sensor. This configuration can present a relation between the output of the force sensor and the output of the myoelectric sensor at each point in time during a period of time in which the output of the force sensor and the output of the myoelectric sensor are produced. Therefore, compared with the case only using the force sensor or the case only using the myoelectric sensor, data can be output which allows data caused by bruxism and data not caused by bruxism to be distinguished from each other more accurately.

A plurality of force sensors (for example, first sensor element 221, second sensor element 222, third sensor element 223) are disposed at the mouthpiece (for example, mouthpiece 21), and the controller (for example, controller 12) outputs data in which outputs of the force sensors can be individually identified (see, for example, FIG. 9). With this configuration, the position in the human mouth where the force is generated can be located more accurately, based on the relation between the arrangement of each of force sensors assumed in the human mouth (see, for example, FIG. 6) and the output of each of the force sensors in the data.

The force sensor (for example, first sensor element 221, second sensor element 222, third sensor element 223) is provided at a flexible substrate (for example, FPC 25), and the flexible substrate is mounted on the mouthpiece (for example, mouthpiece 21). With this configuration, the output of the force sensor can be transmitted via the flexible substrate. The flexibility of the flexible substrate makes it easier to achieve both flexibility for human mouth movement and a configuration for more reliable transmission of output of the force sensor.

The flexible substrate (for example, FPC 25) is adhesively fixed such that a gap is formed between the flexible substrate and the mouthpiece, inside the mouthpiece (for example, mouthpiece 21) (see, for example, FIG. 4). This configuration easily prevents or reduces the force on the mouthpiece from acting directly on the flexible substrate. This configuration also more easily enhances flexibility and engagement of the mouthpiece with protrusions and depressions of the teeth in the human mouth.

The myoelectric sensor (for example, myoelectric sensor 40) includes an electrode (for example, first electrode 42, second electrode 43, and third electrode 44), and gel (for example, conductive gels 42a, 43a, 44a) and double-sided tape (for example, double-sided tape 45 for skin) are applied on an attachment surface of the myoelectric sensor that is provided with the electrode. This configuration facilitates attachment of the myoelectric sensor to the human while further ensuring that the electrode is in close proximity to the human cheek.

The controller (for example, controller 12) causes the myoelectric sensor (for example, myoelectric sensor 40) to operate at a first cycle until the output of the myoelectric sensor exceeds a first threshold (for example, first threshold Th1), and causes the myoelectric sensor to operate at a second cycle after the output of the myoelectric sensor exceeds the first threshold. The second cycle is a higher-frequency cycle than the first cycle. As used herein, the first cycle is, for example, the low-frequency cycle in the description with reference to FIG. 12. As used herein, the second cycle is, for example, the high-frequency cycle in the description with reference to FIG. 12. This configuration gives higher priority to power saving until the output of the myoelectric sensor that may be an output corresponding to bruxism is produced. After the output of the myoelectric sensor that may be an output corresponding to bruxism is produced, more accurate sensing can be performed with more frequent outputs.

The controller (for example, controller 12) causes the force sensor (for example, force sensor 20) to operate at a third cycle until the output of the force sensor exceeds a second threshold (for example, second threshold Th2), and causes the force sensor to operate at a fourth cycle after the output of the force sensor exceeds the second threshold. The fourth cycle is a higher-frequency cycle than the third cycle. As used herein, the third cycle is, for example, the low-frequency cycle in the description with reference to FIG. 13. As used herein, the fourth cycle is, for example, the high-frequency cycle in the description with reference to FIG. 13. This configuration gives higher priority to power saving until the output of the force sensor that may be an output corresponding to bruxism is produced. After the output of the force sensor that may be an output corresponding to bruxism is produced, more accurate sensing can be performed with more frequent outputs.

The detection device 10 also includes a communication circuit (for example, communication circuit 13) that communicates with an external apparatus (for example, terminal device 60). The controller (for example, controller 12) does not cause the communication circuit to operate when neither a first condition nor a second condition is satisfied, and causes the communication circuit to operate when at least one of the first condition and the second condition is satisfied. The first condition is that the output of the myoelectric sensor (for example, myoelectric sensor 40) exceeds a first threshold (for example, first threshold Th1). The second condition is that the output of the force sensor (for example, force sensor 20) exceeds a second threshold (for example, second threshold Th2). This configuration gives higher priority to power saving until the output of at least one of the myoelectric sensor and the force sensor that may be an output corresponding to bruxism is produced. After an output that may be an output corresponding to bruxism is produced, more accurate sensing can be performed with outputs at a higher frequency.

A display (for example, display 63) configured to perform display output in which the output of the force sensor (for example, force sensor 20) is synchronized with the output of the myoelectric sensor (for example, myoelectric sensor 40) can output data that can be visually recognized by a user who can see the display.

In the detection system 100, the display 63 that performs display output such as the graph 63a is provided in the terminal device 60, but such a configuration may be included in the detection device 10. In other words, the detection device 10 may include a display configured to perform display output in which the output of the force sensor 20 is synchronized with the output of the myoelectric sensor 40. In this case, for example, the display may be provided on that surface of the casing 41 of the detection device 10 described with reference to FIG. 2 on which the first electrode 42, the second electrode 43, and the third electrode 44 are not provided. The display coupled through a wire extending from the casing 41 may be included in the configuration of the detection device 10.

The specific configuration of the force sensor 20 is not limited to the one using the piezoelectric effect described above. For example, the specific configuration of the sensor element 22 may be a strain gauge, and a circuit including a Wheatstone bridge may be provided in the casing 41. In this case, strain generated in each of the first sensor element 221, the second sensor element 222, and the third sensor element 223 produces an output representing force. A plurality of sensor elements, such as the first sensor element 221, the second sensor element 222, and the third sensor element 223, are not necessarily provided, and a sensor element in a curved shape along the shape of human teeth may be employed. In addition, the specific configuration of the force sensor 20 may be a resistive force sensor. However, the force sensor 20 is preferably a film-type force sensor.

Other effects brought about by the manners described in the present embodiment that are obvious from the description here or that can be conceived by a person skilled in the art should be understood to be brought about by the present disclosure.

What is claimed is:

1. A detection device comprising:
a force sensor disposed at a mouthpiece;
a myoelectric sensor attachable to a human cheek; and
a controller configured to perform output in which an output of the force sensor is synchronized with an output of the myoelectric sensor.

2. The detection device according to claim 1, wherein
a plurality of force sensors are disposed at the mouthpiece, and
the controller outputs data in which outputs of the force sensors are individually identifiable.

3. The detection device according to claim 1, wherein
the force sensor is disposed at a flexible substrate, and
the flexible substrate is mounted on the mouthpiece.

4. The detection device according to claim 3, wherein the flexible substrate is adhesively fixed such that a gap is formed between the flexible substrate and the mouthpiece, inside the mouthpiece.

5. The detection device according to claim 1, wherein
the myoelectric sensor includes an electrode, and
gel and double-sided tape are applied on an attachment surface of the myoelectric sensor that is provided with the electrode.

6. The detection device according to claim 5, wherein
the controller
causes the myoelectric sensor to operate at a first cycle until the output of the myoelectric sensor exceeds a first threshold, and
causes the myoelectric sensor to operate at a second cycle after the output of the myoelectric sensor exceeds the first threshold, and
the second cycle is a higher-frequency cycle than the first cycle.

7. The detection device according to claim 5, wherein
the controller
causes the force sensor to operate at a third cycle until the output of the force sensor exceeds a second threshold, and
causes the force sensor to operate at a fourth cycle after the output of the force sensor exceeds the second threshold, and
the fourth cycle is a higher-frequency cycle than the third cycle.

8. The detection device according to claim 5, further comprising a communication circuit configured to communicate with an external apparatus, wherein
the controller
does not cause the communication circuit to operate when neither a first condition nor a second condition is satisfied, and
causes the communication circuit to operate when at least one of the first condition and the second condition is satisfied,
the first condition is that the output of the myoelectric sensor exceeds a first threshold, and
the second condition is that the output of the force sensor exceeds a second threshold.

9. The detection device according to claim 1, further comprising a display configured to perform display output in which the output of the force sensor is synchronized with the output of the myoelectric sensor.
